(19) United States
(12) Patent Application Publication
(10) Pub. No.: US 2023/0174938 A1
(43) Pub. Date: Jun. 8, 2023

(54) METHODS AND COMPOSITIONS FOR GENERATING HUMAN ERYTHROID PROGENITOR CELLS

(71) Applicant: Trailhead Biosystems Inc., Cleveland, OH (US)

(72) Inventor: Angelica UELTSCHY, Shaker Heights, OH (US)

(21) Appl. No.: 18/076,254

(22) Filed: Dec. 6, 2022

Related U.S. Application Data
(60) Provisional application No. 63/287,372, filed on Dec. 8, 2021.

(51) Int. Cl. C12N 5/078

(52) U.S. Cl. CPC C12N 5/0641; C12N 2506/11; C12N 2501/2303; C12N 2501/14; C12N 2500/24; C12N 2500/36; C12N 2501/999

(57) ABSTRACT

Methods for inducing human erythroid progenitor cells from hematopoietic stem cells are provided using chemically-defined culture media. Erythroid progenitors generated by the methods include megakaryocyte/erythroid progenitor cells (MEP cells) and CD71+CD235+CD34− erythroid cells, which can be further differentiated into red blood cells. Culture media, isolated cell populations and kits are also provided.

### RELATED APPLICATIONS

This application claims priority to U.S. Provisional Application No. 63/287,372, filed Dec. 8, 2021. The entire contents of which is hereby incorporated by reference.

### BACKGROUND OF THE INVENTION

Hematopoietic stem cells (HSCs) are pluripotent, self-renewing cells that give rise to the entire hematopoietic system. HSCs are rare cells naturally found in bone marrow and umbilical cord blood, and even more rarely in peripheral blood. HSCs are typically defined by the expression, or lack of expression, of particular markers, including expression of CD34 and lack of expression of Lineage-specific markers and CD38 (Lin-CD34+CD38−). The culture conditions under which HSCs are grown determines the differentiation of HSCs to downstream cell lineages of the hematopoietic system. Thus, identification of culture conditions that promote generation of specific hematopoietic lineages are of great interest.

Under appropriate conditions, HSCs can differentiate into blood components including red blood cells and platelets. Given the regular and widespread shortages of blood products for transfusion, as well as concerns with contamination of donated blood with existing and emerging pathogens, the ability to generate blood components in vitro from HSCs is a focus of regenerative medicine.

The earliest protocols for RBC generation from CD34+ HSCs identified interleukin-3 (IL-3), erythropoietin (EPO) and Stem Cell Factor (SCF) as important culture components for erythroid lineage development (see e.g., Giarratana et al. (2011) Blood 118:5071-5079; Kim (2014) Yonsei Med. J. 55:304-309). However, since SCF is an expensive reagent, culture media for generating erythroid progenitors that comprise SCF are expensive relative to media not containing SCF.

Additional protocols for generating red blood cells from CD34+ HSCs have been described that further defined components for specific stages of differentiation. For example, a four-stage protocol for generating erythroid cells over 21 days has been described that utilized culture components including not only IL-3, EPO and SCF, but also thrombopoietin (TPO), granulocyte-macrophage colony-stimulating factor (GM-CSF) and fms-related tyrosine kinase 3 ligand (FL), as well as bovine serum (Zhang et al. (2017) Stem Cells Transl. Med. 6:1698-1709). A serum-free erythroid differentiation protocol has been described that, in addition to using IL-3, EPO and SCF, included dexamethasone, insulin, holotransferrin and bovine serum albumin in the culture media (Uchida et al. (2018) Mol. Ther. Methods Clin. Dev. 9:247-256).

Accordingly, while some progress has been, there remains a need for efficient and robust methods and compositions for generating human erythroid progenitor cells in culture from human hematopoietic stem cells.

### SUMMARY OF THE INVENTION

This disclosure provides methods for generating human erythroid progenitor cells from human CD34+ hematopoietic stem cells (HSCs), e.g., from umbilical cord blood or bone marrow cells, using chemically-defined culture media. The disclosure provides a two-stage protocol that allows for obtention of GATA1+ megakaryocyte/erythroid progenitor cells in as little as four days of culture and the obtention of CD71+CD235+CD34− erythroid progenitor cells in as little as nine days of culture. Thus, the methods of the disclosure can be used to obtain progenitor cells for both the megakaryocyte/platelet lineage and the erythroid lineage, as well as more differentiated progenitor cells along the erythroid lineage that can be further differentiated into red blood cells.

The culture media of the disclosure comprises small molecule agents that either agonize or antagonize particular signaling pathway in stem cells such that differentiation of the HSCs along the erythroid lineage is promoted, leading to expression of erythroid-associated biomarkers. The methods of the disclosure have the advantage that they significantly shorten the time needed to obtain erythroid progenitors, as well as decreasing the associated costs by using less expensive reagents than earlier protocols. For example, the culture media of the disclosure do not require the use of the costly Stem Cell Factor (SCF) reagent. Moreover, the use of small molecule agents in the culture media allows for precise control of the culture components.

Accordingly, in one aspect, the disclosure pertains to a method of generating human GATA1+ megakaryocyte/erythroid progenitor (MEP) cells comprising: culturing human CD34+ hematopoietic stem cells (HSCs) in a culture media comprising an IL3R pathway agonist, a TGFβ pathway agonist, an AHR pathway antagonist, a RET pathway antagonist and an AKT pathway antagonist on days 0-4 to obtain human GATA1+ MEP cells.

In an embodiment, the human GATA1+ MEP cells are further cultured on days 4-9 in a culture media comprising an AHR antagonist, an iron source, an EPOR agonist, an AMPK agonist and a lipid source to obtain human CD71+CD235+CD34− erythroid progenitor cells.

In an embodiment, the HSCs are from umbilical cord blood. In an embodiment, the HSCs are from bone marrow. In an embodiment, the HSCs are from peripheral blood.

In an embodiment, the IL3R pathway agonist is IL-3. In an embodiment, IL-3 is present in the culture media at a concentration within a range of 5-15 ng/ml. In an embodiment, IL-3 is present in the culture media at a concentration of 10 ng/ml.

In an embodiment, the TGFβ pathway agonist is selected from the group consisting of alantolactone, Activin A, TFGB1, Nodal, and combinations thereof. In an embodiment, the TGFβ pathway agonist is present in the culture media at a concentration within a range of 500-1000 nM. In an embodiment, the TGFβ pathway agonist is alantolactone, which is present in the culture media at a concentration of 750 nM.

In an embodiment, the AHR pathway antagonist is selected from the group consisting of SR1, GNF351, AHR antagonist 5 hemimaleate, AHR antagonist 1, PDM2, BAY 2416964, CH-223191, AHR antagonist 2, AHR antagonist 4, and combinations thereof. In an embodiment, the AHR pathway antagonist is present in the culture media at a concentration within a range 500-1000 nM. In an embodiment, the AHR pathway antagonist is SR1, which is present in the culture media at a concentration of 750 nM.

In an embodiment, the RET pathway antagonist is selected from the group consisting of RETki, Lenvatinib, Regorafenib, Pralsetinib, Selpertinib, Lenvatinib mesylate, RET-IN-4, RPI-1, JNJ38158471, Amuvatinib, TG101209, Regorafenib Hydrochloride, Ilorasertib hydrochloride, AST487, PF477736, BBT594, AD80, GSK3179106, SPP86, RET-IN-3, WF-47-JS03, RET V804M-IN-1, Trans-pralsetinib, PZ1, Regorafenib D3, RET-IN-1, ML786 dihydrochloride, WHI-P180 hydrochloride, and combinations thereof. In an embodiment, the RET pathway antagonist is present in the culture media at a concentration within a range of 500-1000 nM. In an embodiment, the RET pathway agonist is RETki, which is present in the culture media at a concentration of 750 nM.

In an embodiment, the AKT pathway antagonist is selected from the group consisting of MK2206, GSK690693, Perifosine (KRX-0401), Ipatasertib (GDC-0068), Capivasertib (AZD5363), PF-04691502, AT 7867, Triciribine (NSC154020), ARQ751, Miransertib (ab235550), Borussertib, Cerisertib, Akti1/2, CCT128930, A 674563, PHT 427, Miltefosine, AT 13148, ML 9, BAY 1125976, Oridonin, TIC10, Pectolinarin, Acti IV, 10-DEBC, API-1, SC 66, FPA 124, API-2, Urolithin A, and combinations thereof. In an embodiment, the AKT pathway antagonist is present in the culture media at a concentration within a range of 50-150 nM. In an embodiment, the AKT pathway antagonist is MK2206, which is present in the culture media at a concentration of 100 nM.

In an embodiment, the iron source is selected from the group consisting of holotransferrin, FeIII_EDTA, Optferrin, FeSO4, Ferrous nitrate, lactoferrin, ferritin, and combinations thereof. In an embodiment, the iron source is present in the culture media at a concentration within a range of 150-250 ug/ml. In an embodiment, the iron source is holotransferrin, which is present in the culture media at a concentration of 200 ug/ml.

In an embodiment, the EPOR pathway agonist is selected from the group consisting of EPO, EPO analogs, and combinations thereof. In an embodiment, the EPOR pathway agonist is EPO. In an embodiment, the EPOR pathway agonist is EPO, which is present in the culture media at a concentration of 2 U/ml.

In an embodiment, the AMPK pathway agonist is selected from the group consisting of AICAR, Metformin, BC1618, Malvidin-3-O-arabinoside chloride, A-769662, MK8722, Bempedoic acid, AICAR phosphate, Phenformin hydrochloride, EX229, gingerol, Kazinol B, PF06409577, Flufenamic acid, GSK621, Urolithin B, MK3903, chitosan oligosaccharide, palmitelaidic acid, O-304, Amarogentin, 7-Methoxyisoflavone, EB-3D, Buformin hydrochloride, Platycodin D, ZLN024 hydrochloride, Danthron, Ampkinone, ginkolide C, Gomisin J, Demethylenebernerine, ASP4132, IM156, Vacarin, MOTS-c(human) acetate, Kahweol, AMPK activator 4, Marein, Euphorbiasteroid, Cimiracemoside C, Metformin D6 hydrochloride, MT6378, RSVA405, Nepodin, 3α-Hydroxymogrol, AMPK activator 1, YLF-466D, Buformin, IQZ23, Galegine hydrochloride, Karanjin, COH-SR4, HL271, ZLN024, EB-3D, and combinations thereof. In an embodiment, the AMPK pathway agonist is present in the culture media at a concentration within a range of 50-150 uM. In an embodiment, the AMPK pathway agonist is AICAR, which is present in the culture media at a concentration of 100 uM.

In an embodiment, the lipid source is selected from the group consisting of Albumax, free fatty acids, lysophosphatidylcholine triacylglycerides, phosphatidylcholine, phosphatidic acid, cholesterol, sphingomyelin, knockout serum replacement, Lipid Mixture 1™, Chemically Defined Lipid Concentrate™, bovine serum albumin, human serum albumin, and combinations thereof. In an embodiment, the lipid source is Albumax. In an embodiment, the lipid source is Albumax, which is present in the culture media at a concentration of 0.5%.

In another aspect, the disclosure pertains to a method of generating human CD71+CD235+CD34− erythroid progenitor cells, the method comprising:

(a) culturing human CD34+ hematopoietic stem cells (HSCs) in a culture media comprising an IL3R pathway agonist, a TGFβ pathway agonist, an AHR pathway antagonist, a RET pathway antagonist and an AKT pathway antagonist on days 0-4 to obtain human GATA1+ MEP cells; and

(b) further culturing the human GATA1+ MEP cells in a culture media comprising an AHR antagonist, an iron source, an EPOR agonist, an AMPK agonist and a lipid source on days 4-9 to obtain human CD71+CD235+CD34− erythroid progenitor cells.

Non-limiting exemplary reagents and concentrations include those listed above. In an embodiment, in step (a) the IL3R pathway agonist is IL-3, the TGFβ pathway antagonist is alantolactone, the AHR pathway antagonist is SR1, the RET pathway antagonist is RETki, and the AKT pathway antagonist is MK2206. In an embodiment, in step (a) IL-3 is present in the culture media at a concentration of 10 ng/ml, alantolactone is present in the culture media at a concentration of 750 nM, SR1 is present in the culture media at a concentration of 750 nM, RETki is present in the culture media at a concentration of 750 nM, and MK2206 is present in the culture media at a concentration of 100 nM.

In an embodiment, in step (b) the AHR pathway antagonist is SR1, the iron source is holotransferrin, the EPOR pathway agonist is EPO, the AMPK pathway agonist is AICAR, and the lipid source is Albumax. In an embodiment, in step (b) SR1 is present in the culture media at a concentration of 750 nM, holotransferrin is present in the culture media at a concentration of 200 ug/ml, EPO is present in the culture media at a concentration of 2 U/ml, AICAR is present in the culture media at a concentration of 100 uM, and Albumax is present in the culture at a concentration of 0.5%.

In another aspect, the disclosure pertains to culture media for generating human erythroid progenitor cells. In an embodiment, the disclosure provides a culture media for obtaining human GATA1+ megakaryocyte/erythroid progenitor (MEP) cells comprising an IL3R pathway agonist, a TGFβ pathway agonist, an AHR pathway antagonist, a RET pathway antagonist and an AKT pathway antagonist. In an embodiment, the disclosure provides a culture media for obtaining human CD71+CD235+CD34− erythroid progenitor cells comprising an AHR antagonist, an iron source, an EPOR agonist, an AMPK agonist and a lipid source.

In another aspect, the disclosure pertains to isolated cell cultures of human erythroid progenitor cells. In an embodiment, the disclosure provides an isolated cell culture of human GATA1+ megakaryocyte/erythroid progenitor (MEP) cells, the culture comprising human GATA1+ MEP cells cultured in a culture media comprising an IL3R pathway agonist, a TGFβ pathway agonist, an AHR pathway antagonist, a RET pathway antagonist and an AKT pathway antagonist. In an embodiment, the disclosure provides an isolated cell culture of human CD71+CD235+CD34− erythroid progenitor cells, the culture comprising human CD71+CD235+CD34− erythroid progenitor cells cultured in a culture media comprising an AHR antagonist, an iron source, an EPOR agonist, an AMPK agonist and a lipid source.

Human erythroid progenitor cells generated by the methods of the disclosure are also provided. In an embodiment, the disclosure pertains to a human GATA1+ megakaryocyte/erythroid progenitor (MEP) cells generated by a method of the disclosure. In an embodiment, the disclosure pertains to a human CD71+CD235+CD34− human erythroid progenitor cells generated by a method of the disclosure.

Other features and advantages of the invention will be apparent from the following detailed description and claims.

### BRIEF DESCRIPTION OF THE DRAWINGS

FIG. 1 shows results from an HD-DoE model of an 8-factor experiment optimized for maximum expression of GATA1. The upper section of the model shows the prediction of expression level of pre-selected 53 genes when optimized for GATA1. The lower section of the model shows the effectors that were tested in this model and their contribution to maximum expression of GATA1. The value column refers to required concentration of each effector to mimic the model.

FIG. 2 shows results from an HD-DoE model of an 6-factor experiment optimized for maximum expression of GATA1. The upper section of the model shows the prediction of expression level of pre-selected 53 genes when optimized for GATA1. The lower section of the model shows the effectors that were tested in this model and their contribution to maximum expression of GATA1. The value column refers to required concentration of each effector to mimic the model.

FIG. 3 shows results from an HD-DoE model of an 12-factor experiment optimized for maximum expression of HBA1. The upper section of the model shows the prediction of expression level of pre-selected 53 genes when optimized for HBA1. The lower section of the model shows the effectors that were tested in this model and their contribution to maximum expression of HBA1. The value column refers to required concentration of each effector to mimic the model.

FIGS. 4A-4B shows the dynamic profile of expression levels of GATA1, GATA2, CD36 and TFRC genes relative to the concentration of 1 validated effector tested for the MEP cell. FIG. 4A shows the expression level of genes of interest in the presence of all three finalized effectors. FIG. 4B shows the expression level of genes of interest in the absence of one finalized effector at a time while the others remained present. The positive impact of the effectors on gene expression and their factor contribution is shown by the slope of the plots for each effector.

FIGS. 5A-5B shows the dynamic profile of expression levels of GATA1, HBA1, GYPA and TFRC genes relative to the concentration of 1 validated effector tested for erythroid conversion. FIG. 5A shows the expression level of genes of interest in the presence of all six finalized effectors. FIG. 5B shows the expression level of genes of interest in the absence of one finalized effector at a time while the others remained present. The positive impact of the effectors on gene expression and their factor contribution is shown by the slope of the plots for each effector.

FIGS. 6A-6B show the results of flow cytometry analyses of cord blood CD34+ cells grown in stage 1 media or literature media for 4 or 5 days. FIG. 6A shows the results for cells stained with antibodies for CD71, CD235 (glycogen) and FVS700 a live and dead marker, to exclude dead cells from the analysis. FIG. 6B shows the mean fluorescent intensity (MFI) at day 4 and 5 of CD71+ cells grown in stage 1 media and culture media.

FIGS. 7A-7B show the results of flow cytometry analyses of cord blood CD34+ cells grown in stage 1 media (red) or literature media (blue) for 4 or 5 days. Results are shown for cells stained with antibodies for CD71, CD34, CD38, CD123, CD45RA, CD41 and FVS700, and a live and dead marker, to exclude dead cells from the analysis.

FIGS. 8A-8C show the results of flow cytometry analyses of cord blood CD34+ cells grown in stage 1 media or literature media for 4 days, followed by growth on stage 2 media or literature media for 3, 5 or 7 days. Cells were stained with antibodies for CD71, CD235 (glycophorin A and B) and FVS700, a live and dead marker, to exclude dead cells from the analysis. FIG. 8A shows the results for culture in stage 2 media for 3 days. FIG. 8B shows the results for culture in stage 2 media for 5 days. FIG. 8C shows the results for culture in stage 2 media for 7 days.

FIGS. 9A-9C show the results of flow cytometry analyses of cord blood CD34+ cells grown in stage 1 media or literature media for 4 days, followed by growth on stage 2 media or literature media for 3, 5 or 7 days. Cells were stained for DNA using DRAQ5. FIG. 9A shows the results for culture in stage 2 media for 3 days. FIG. 9B shows the results for culture in stage 2 media for 5 days. FIG. 9C shows the results for culture in stage 2 media for 7 days.

FIG. 10 shows the results of flow cytometry analyses of cord blood CD34+ cells grown in stage 1 media for 4 days, followed by growth on stage 2 media for 5 days using two different basal media. Stemline 2 media and an exemplary basal media were compared. Cells were stained with antibodies for CD71, CD235 (glycophorin A and B) and FVS700, a live and dead marker, to exclude dead cells from the analysis. Cell number was evaluated in the culture after growing cells on stage 2 media for 5 days.

FIGS. 11A-11B shows contour plots showing HBA expression in different concentrations of cholesterol supplement and holo-transferrin (FIG. 11A) and cholesterol supplement and EPO (FIG. 11B). The red area of the plot indicates maximal HBA expression, while the blue indicates lowest HBA expression. This analysis indicates that cholesterol supplementation and higher concentrations of EPO and holo-transferrin are optimal for maximal HBA expression in the system.

FIG. 12 shows results from an HD-DoE model of a 12-factor experiment optimized for maximum expression of HBG2, a red blood cell marker. The upper section of the model shows the prediction of expression level of pre-selected 53 genes when optimized for HBG2. The lower section of the model shows the effectors that were tested in this model and their contribution to maximum expression of HBG2. The value column refers to required concentration of each effector to mimic the model. This model shows that HSCs grown in stage 1 media to generate MEP cells leads to cells with the potential to differentiate to the erythroid lineage.

FIG. 13 shows results from an HD-DoE model of an 8-factor experiment optimized for maximum expression of PF4, a megakaryocyte/platelet marker. The upper section of the model shows the prediction of expression level of pre-selected 53 genes when optimized for PF4. The lower section of the model shows the effectors that were tested in this model and their contribution to maximum expression of PF4. The value column refers to required concentration of each effector to mimic the model. This model shows that HSCs grown in stage 1 media to generate MEP cells leads to cells with the potential to differentiate to the platelet lineage.

FIG. 14 is a schematic diagram of a representative culture method of the disclosure.

### DETAILED DESCRIPTION OF THE INVENTION

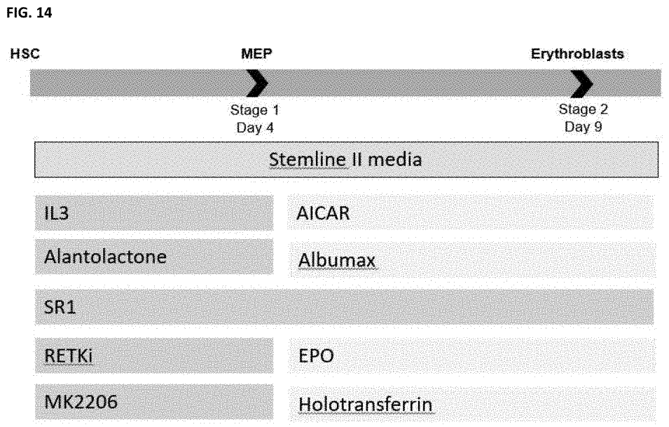

Described herein are methodologies and compositions that allow for generation of human erythroid progenitor cells from human CD34+ hematopoietic stem cells (HSCs) under chemically-defined culture conditions using a small molecule based approach. As described in Example 1, a High-Dimensional Design of Experiments (HD-DoE) approach was used to simultaneously test multiple process inputs (e.g., small molecule agonists or antagonists) on output responses, such as gene expression. These experiments allowed for the identification of chemically-defined culture media, comprising agonists and/or antagonists of particular signaling pathways, that is sufficient to generate erythroid progenitors from HSCs in a very short amount of time in a two stage protocol. The first stage of the protocol generates GATA1+ Megakaryocyte/Erythroid Progenitor (MEP) cells from CD34+ HSCs. The second stage of the protocol generates CD71+CD235+CD34− erythroid progenitor cells from the GATA1+ MEP cells. The optimized two-stage culture media was further validated by a factor criticality analysis, which examined the effects of eliminating individual agonist or antagonist agents, as described in Example 2. Flow cytometry analysis further confirmed the phenotype of the cells generated by the differentiation protocol, as described in Example 3. Furthermore, the differentiation potential of the Megakaryocyte/Erythroid Progenitor (MEP) cell generated by the methods of the disclosure was examined in Example 4. A representative culture method of the disclosure is shown in the schematic diagram of FIG. 14.

Various aspects of the invention are described in further detail in the following subsections.

### I. Cells

The starting cells used in the cultures of the disclosure are human CD34+ hematopoietic stem cells. As used herein, the term “hematopoietic stem cell” (abbreviated as HSC) refers to a stem cell that has the capacity to differentiate into a variety of different hematopoietic cell types. CD34 is a transmembrane phosphoglycoprotein that has been established in the art as a surface marker for HSCs. Human HSCs are readily obtainable from available sources, including human umbilical cord blood, adult bone marrow and peripheral blood. HSCs include both long term HSCs (LT-HSCs) and short term HSCs (ST-HSCs).

Long term HSCs (LT-HSCs) are HSCs that are found in the bone marrow or cord blood that, through a process of asymmetric cell division, can self-renew to sustain the stem cell pool or differentiate into short-term HSCs (ST-HSCs) or lineage-restricted progenitors that undergo extensive proliferation and differentiation to produce terminally differentiated cells of the blood lineage. It is believed that LT-HSCs are enriched on the fraction of Lin-CD34+CD38−CD45RA−CD90+ cells. LT-HSCs are quiescent and slow to divide in culture, taking up to 80 hours to first cell division (Cheung and Rando (2013) Nat. Rev. Mol. Cell Biol. 14:329-340). In contrast, short term HSCs (ST-HSCs) by definition have limited self-renewal capacity, generally described as giving rise to lymphohematopoiesis for 4-12 weeks before senescence.

In an embodiment, the HSCs express CD34 (CD34+). In an embodiment, the HSCs lack expression of the marker Lineage (Lin−). In an embodiment, the HSCs lack expression of CD38 (CD38−). In an embodiment, the HSCs lack expression of CD45RA (CD45RA−). In an embodiment, the HSCs express CD90 (CD90+). In an embodiment, the HSCs are Lin−CD34+CD38−CD45RA−CD90+ cells.

In embodiments, the HSCs express one or more genes associated with the HSC phenotype (also referred to herein as HSC-associated genetic markers), non-limiting examples of which include CHRBP, Mecom, Meg3, HOPX, LMO2, CD34, TAL1 and GATA2.

### II. Culture Media Components

The method of the disclosure for generating human erythroid progenitor cells from CD34+ HSCs comprise culturing the human CD34+ HSCs in a culture media comprising specific agonist and/or antagonists of cellular receptors and/or signaling pathways. In certain embodiments, the culture media lacks exogenously added serum, lacks exogenously added bovine serum albumin and/or lacks Stem Cell Factor (SCF) (i.e., the media does not comprise SCF).

In an embodiment, the disclosure provides a culture media for obtaining human GATA1+ megakaryocyte/erythroid progenitor (MEP) cells comprising an IL3R pathway agonist, a TGFβ pathway agonist, an AHR pathway antagonist, a RET pathway antagonist and an AKT pathway antagonist. In an embodiment, the disclosure provides a culture media for obtaining human CD71+CD235+CD34− erythroid progenitor cells comprising an AHR antagonist, an iron source, an EPOR agonist, an AMPK agonist and a lipid source.

As described in Example 1, a culture media comprising an IL3R pathway agonist, a TGFβ pathway agonist, an AHR pathway antagonist, a RET pathway antagonist and an AKT pathway antagonist was sufficient to generate GATA1+ MEP cells from CD34+ HSCs in as little as four days. Furthermore, further culture for five more days in a culture media comprising an AHR antagonist, an iron source, an EPOR agonist, an AMPK agonist and a lipid source was sufficient to generate CD71+CD235+CD34− erythroid progenitor cells from the GATA1+ MEP cells.

As used herein, an “agonist” of a cellular receptor or signaling pathway is intended to refer to an agent that stimulates (upregulates) the cellular receptor or signaling pathway. Stimulation of the cellular signaling pathway can be initiated extracellularly, for example by use of an agonist that activates a cell surface receptor involved in the signaling pathway (e.g., the agonist can be a receptor ligand). Additionally or alternatively, stimulation of cellular signaling can be initiated intracellularly, for example by use of a small molecule agonist that interacts intracellularly with a component(s) of the signaling pathway.

As used herein, an “antagonist” of a cellular signaling pathway is intended to refer to an agent that inhibits (downregulates) the cellular signaling pathway. Inhibition of the cellular signaling pathway can be initiated extracellularly, for example by use of an antagonist that blocks a cell surface receptor involved in the signaling pathway. Additionally or alternatively, inhibition of cellular signaling can be initiated intracellularly, for example by use of a small molecule antagonist that interacts intracellularly with a component(s) of the signaling pathway.

IL3R pathway agonists, TGFβ pathway agonists, AHR pathway antagonists, RET pathway antagonists, AKT pathway antagonists, iron sources, EPOR agonists, AMPK agonists and lipid sources are known in the art and commercially available. They are used in the culture media at a concentration effective to achieve the desired outcome, e.g., generation of erythroid progenitor cells expressing markers of interest. Non-limiting examples of suitable agonist and antagonists agents, and effective concentration ranges, are described further below.

Agonists of the IL3R pathway include agents, molecules, compounds, or substances capable of stimulating (activating) the IL3R signaling pathway. In an embodiment, the IL3R pathway agonist is IL3 or a IL3R-binding analog thereof. In another embodiment, the IL3R pathway agonist is IL-3. In an embodiment, the IL3R pathway agonist is IL-3, which is present in the media at a concentration range of 5-15 ng/ml, 6-14 ng/ml, 7-13 ng/ml or 8-12 ng/ml. In an embodiment, the IL3R pathway agonist is IL-3, which is present in the media at a concentration of 10 ng/ml.

Agonists of the TGFβ pathway include agents, molecules, compounds, or substances capable of stimulating (activating) the TGFβ signaling pathway. In an embodiment, the TGFβ pathway agonist is selected from the group consisting of alantolactone, Activin A, TGFB1, Nodal, and combinations thereof. In an embodiment, the TGFβ pathway agonist is Activin A. In another embodiment, the TGFβ pathway agonist is alantolactone. In an embodiment, the TGFβ pathway agonist is alantolactone, which is present in the media at a concentration range of 500-1000 nM, 600-900 nM or 700-800 nM. In an embodiment, the TGFβ pathway agonist is alantolactone, which is present in the media at a concentration of 750 nM.

Antagonists of the AHR (aryl hydrocarbon receptor) pathway include agents, molecules, compounds, or substances capable of inhibiting (downregulating) the AHR signaling pathway. In one embodiment, the AHR pathway antagonist is selected from the group consisting of SR1, GNF351, AHR antagonist 5 hemimaleate, AHR antagonist 1, PDM2, BAY 2416964, CH-223191, AHR antagonist 2, AHR antagonist 4, and combinations thereof. In an embodiment, the AHR pathway antagonist is present in the culture media at a concentration within a range 500-1000 nM, 600-900 nM or 700-800 nM. In an embodiment, the AHR pathway antagonist is SR1, which is present in the culture media at a concentration within a range 500-1000 nM, 600-900 nM or 700-800 nM. In an embodiment, the AHR pathway antagonist is SR1, which is present in the culture media at a concentration of 750 nM.

Antagonists of the RET (rearranged during transfection) pathway include agents, molecules, compounds, or substances capable of inhibiting (downregulating) the RET signaling pathway. In one embodiment, the RET pathway antagonist is selected from the group consisting of RETki, Lenvatinib, Regorafenib, Pralsetinib, Selpertinib, Lenvatinib mesylate, RET-IN-4, RPI-1, JNJ38158471, Amuvatinib, TG101209, Regorafenib Hydrochloride, Ilorasertib hydrochloride, AST487, PF477736, BBT594, AD80, GSK3179106, SPP86, RET-IN-3, WF-47-JS03, RET V804M-IN-1, Trans-pralsetinib, PZ1, Regorafenib D3, RET-IN-1, ML786 dihydrochloride, WHI-P180 hydrochloride, and combinations thereof. In an embodiment, the RET pathway antagonist is present in the culture media at a concentration within a range 500-1000 nM, 600-900 nM or 700-800 nM. In an embodiment, the RET pathway antagonist is RETki, which is present in the culture media at a concentration within a range 500-1000 nM, 600-900 nM or 700-800 nM. In an embodiment, the RET pathway antagonist is RETki, which is present in the culture media at a concentration of 750 nM.

Antagonists of the AKT pathway include agents, molecules, compounds, or substances capable of inhibiting (downregulating) the AKT signaling pathway. In one embodiment, the AKT pathway antagonist is selected from the group consisting of MK2206, GSK690693, Perifosine (KRX-0401), Ipatasertib (GDC-0068), Capivasertib (AZD5363), PF-04691502, AT 7867, Triciribine (NSC154020), ARQ751, Miransertib (ab235550), Borussertib, Cerisertib, Akti1/2, CCT128930, A 674563, PHT 427, Miltefosine, AT 13148, ML 9, BAY 1125976, Oridonin, TIC10, Pectolinarin, Acti IV, 10-DEBC, API-1, SC 66, FPA 124, API-2, Urolithin A, and combinations thereof. In an embodiment, the AKT pathway antagonist is present in the culture media at a concentration within a range 500-1000 nM, 600-900 nM or 700-800 nM. In an embodiment, the AKT pathway antagonist is MK2206, which is present in the culture media at a concentration within a range 500-1000 nM, 600-900 nM or 700-800 nM. In an embodiment, the AKT pathway antagonist is MK2206, which is present in the culture media at a concentration of 750 nM.

Non-limiting exemplary embodiments of iron sources include holotransferrin, FeIII_EDTA, Optferrin, FeSO4, Ferrous nitrate, and combinations thereof. In an embodiment, the iron source is present in the culture media at a concentration within a range of 100-300 ug/ml, 150-250 ug/ml or 175-225 ug/ml. In an embodiment, the iron source is holotransferrin, which is present in the culture media at a concentration within a range of 100-300 ug/ml, 150-250 ug/ml or 175-225 ug/ml. In an embodiment, the iron source is holotransferrin, which is present in the culture media at a concentration of 200 ug/ml.

Agonists of the EPOR pathway include agents, molecules, compounds, or substances capable of stimulating (activating) the erythropoietin receptor (EPOR) signaling pathway. In an embodiment, the EPOR pathway agonist is EPO or a EPOR-binding analog thereof. In another embodiment, the EPOR pathway agonist is EPO. In an embodiment, the EPOR pathway agonist is EPO, which is present in the media at a concentration range of 1-3 U/ml, 1.5-2.5 U/ml or 1.75-2.25 U/ml. In an embodiment, the EPOR pathway agonist is EPO, which is present in the media at a concentration of 2 U/ml.

Agonist of the AMPK (AMP activated protein kinase) pathway include agents, molecules, compounds, or substances capable of stimulating (activating) the AMPK signaling pathway. In one embodiment, the AMPK pathway agonist is selected from the group consisting of AICAR, Metformin, BC1618, Malvidin-3-O-arabinoside chloride, A-769662, MK8722, Bempedoic acid, AICAR phosphate, Phenformin hydrochloride, EX229, gingerol, Kazinol B, PF06409577, Flufenamic acid, GSK621, Urolithin B, MK3903, chitosan oligosaccharide, palmitelaidic acid, O-304, Amarogentin, 7-Methoxyisoflavone, EB-3D, Buformin hydrochloride, Platycodin D, ZLN024 hydrochloride, Danthron, Ampkinone, ginkolide C, Gomisin J, Demethylenebernerine, ASP4132, IM156, Vacarin, MOTS-c(human) acetate, Kahweol, AMPK activator 4, Marein, Euphorbiasteroid, Cimiracemoside C, Metformin D6 hydrochloride, MT6378, RSVA405, Nepodin, 3α-Hydroxymogrol, AMPK activator 1, YLF-466D, Buformin, IQZ23, Galegine hydrochloride, Karanjin, COH-SR4, HL271, ZLN024, EB-3D, and combinations thereof. In an embodiment, the AMPK pathway agonist is present in the culture media at a concentration within a range of 50-250 uM, 50-150 uM or 75-125 uM. In an embodiment, the AMP pathway agonistt is AICAR, which is present in the culture media at a concentration within a range of 50-250 uM, 50-150 uM or 75-125 uM. In an embodiment, the AMP pathway agonist is AICAR, which is present in the culture media at a concentration of 100 uM.

Non-limiting exemplary embodiments of lipid sources include Albumax, free fatty acids, lysophosphatidylcholine triacylglycerides, phosphatidylcholine, phosphatidic acid, cholesterol, sphingomyelin, knockout serum replacement, Lipid Mixture 1™ (Sigma-Aldrich), Chemically Defined Lipid Concentrate™ (ThermoFisher Scientific), and combinations thereof. In an embodiment, the lipid source is Albumax. In an embodiment, the lipid source is Albumax, which is present in the culture media at a concentration within a range of 0.1-1.0%, 0.25-0.75% or 0.4-0.6%. In an embodiment, the lipid source is Albumax, which is present in the culture media at a concentration of 0.5%.

### III. Culture Conditions

In combination with the chemically-defined and optimized culture media described in subsection II above, the methods of generating human erythroid progenitor cells from CD34+ HSCs of the disclosure utilize standard culture conditions established in the art for cell culture. For example, cells can be cultured at 37° C. and under 5% O₂ and 5% CO₂ conditions. A basal media can be used as the starting media to which supplemental agents can be added. For example, in an embodiment, the commercially available Stemline® II Hematopoietic Stem Cell Expansion Media (Sigma-Aldrich) can be used as basal media. In another embodiment, the commercially available StemSpan™ SFEM II media (STEMCELL Technologies) can be used as basal media. Other non-limiting examples of suitable basal media include X-VIVO™ 15 Serum-free Hematopoietic Cell Medium (Lonza Bioscience), StemMACS™ HSC Expansion Media (Miltenyi Biotec) and StemPro™-34 SFM (ThermoFisher Scientific; catalog no. 10639011). Moreover, suitable serum free basal media can be developed by the ordinarily skilled artisan using reagents established in the arts. Cells can be cultured in standard culture vessels or plates, such as culture dishes, culture flasks or 96-well plates.

The starting CD34+ HSCs can be obtained by methodologies established in the art. Sources of human CD34+ HSCs include umbilical cord blood, peripheral blood, and bone marrow. CD34+ HSC can be obtained, for example, by standard magnetic enrichment.

In various embodiments of the methods of the disclosure, the starting CD34+ HSCs are cultured in specified culture media for sufficient time to generate cells expressing one or more biomarkers of the resultant cells of interest. The starting HSCs express the CD34 marker. Non-limiting examples of additional HSC-associated genetic markers include CHRBP, Mecom, Meg3, HOPX, LMO2, CD34, TAL1 and GATA2.

In embodiments, the CD34+ HSCs are cultured in stage 1 media for sufficient time to generate Megakaryocyte/Erythroid Progenitor (MEP) cells, which can be identified based on expression of one or more MEP-associated biomarkers. Non-limiting examples of MEP-associated biomarkers include GATA1, TFRC, TAL1 and GATA2. In various embodiments, the MEP cells may express at least one, at least two, at least three or at least four MEP-associated biomarkers. In an embodiment, cells are cultured for sufficient time to increase the expression level of at least one MEP-associated biomarker by at least 10%, 20%, 30%, 40%, 50%, 60%, 70%, 80%, 90% or 100% as compared to the starting cell population. The level of expression of genetic markers in the cultured cells can be measured by techniques available in the art (e.g., RNAseq analysis). In an embodiment, the MEP cells are GATA1+. In an embodiment, the CD34+ HSCs are cultured in stage 1 media for sufficient time to generate GATA1+ MEP cells, wherein less than 10% of the cells in the culture are positive for CD41 and CD235.

In embodiments, the GATA1+ MEP cells are cultured in stage 2 media for sufficient time to generate erythroid lineage-committed progenitor cells, which can be identified based on expression of one or more erythroid lineage-associated biomarkers. In embodiments, the erythroid lineage committed progenitor cells are CD71+ and CD235+, as well as being CD34 negative (CD34−). Non-limiting examples of additional erythroid lineage-associated biomarkers include HBA1, CD36, TFRC, PRG2, HBB, HBG2, ALAD, ALAS2, CA2, GYPA, GYPB, GATA1, KLF1 and TFRC. In various embodiments, the erythroid progenitor cells may express at least one, at least two, at least three or at least four, at least five or more erythroid lineage-associated biomarkers. In an embodiment, cells are cultured for sufficient time to increase the expression level of at least one erythroid lineage-associated biomarker by at least 10%, 20%, 30%, 40%, 50%, 60%, 70%, 80%, 90% or 100% as compared to the starting cell population. The level of expression of genetic markers in the cultured cells can be measured by techniques available in the art (e.g., RNAseq analysis).

In an embodiment, cells are cultured in stage 1 media on days 0-4 of culture or for at least 4 days or at least 96 hours. In an embodiment, cells are cultured in stage 2 media on days 5-9 of culture or for at least 5 days (following stage 1 culture) or for at least 120 hours (following stage 1 culture).

The culture media typically is changed regularly to fresh media. For example, in various embodiments, media is changed every 24, 48 or 72 hours.

### IV. Uses

The methods and compositions of the disclosure for generating human erythroid progenitor cells allow for efficient and robust availability of these cell populations for a variety of uses. For example, the methods and compositions can be used in the study of erythroid cell development and differentiation, including biology to assist in the understanding of erythroid-related diseases and disorders. For example, the erythroid progenitors generated using the methods of the disclosure can be further purified according to methods established in the art using agents that bind to surface markers expressed on the cells.

Additionally, erythroid progenitors according to the methods of the disclosure are contemplated for use generating blood components for transfusion. For example, the MEP cells of the disclosure can be further differentiated into platelets, which can be used for transfusion in subjects in need thereof. Similarly, the MEP cells and CD71+CD235+CD34− erythroid progenitor cells can be further differentiated into red blood cells, which can be used for transfusion in subjects in need thereof. Accordingly, the methods and compositions of the disclosure can also be used in connection with the treatment of various hematopoietic diseases and disorders that require transfusion of platelets and/or RBCs.

### V. Compositions

In other aspects, the disclosure provides compositions related to the methods of generating erythroid progenitors, including culture media and isolated cell cultures.

In one aspect, the disclosure provides a culture media for obtaining human GATA1+ megakaryocyte/erythroid progenitor (MEP) cells comprising an IL3R pathway agonist, a TGFβ pathway agonist, an AHR pathway antagonist, a RET pathway antagonist and an AKT pathway antagonist. In another aspect, the disclosure provides a culture media for obtaining human CD71+CD235+CD34− erythroid progenitor cells comprising an AHR antagonist, an iron source, an EPOR agonist, an AMPK agonist and a lipid source. Non-limiting examples of suitable agents, and concentrations therefor, include those described in subsection II above.

In another aspect, the disclosure provides an isolated cell culture of human GATA1+ megakaryocyte/erythroid progenitor (MEP) cells, the culture comprising human GATA1+ MEP cells cultured in a culture media comprising an IL3R pathway agonist, a TGFβ pathway agonist, an AHR pathway antagonist, a RET pathway antagonist and an AKT pathway antagonist. In another aspect, the disclosure provides an isolated cell culture of human CD71+CD235+CD34− erythroid progenitor cells, the culture comprising human CD71+CD235+CD34− erythroid progenitor cells cultured in a culture media comprising an AHR antagonist, an iron source, an EPOR agonist, an AMPK agonist and a lipid source. Non-limiting examples of suitable agents, and concentrations therefor, include those described in subsection II above.

In yet another aspect, the disclosure provides erythroid progenitor cells generated by a method of the disclosure. In an embodiment, the disclosure provides human GATA1+ megakaryocyte/erythroid progenitor (MEP) cells generated by a method of the disclosure, as described herein. In an embodiment, the disclosure provides human CD71+CD235+CD34− human erythroid progenitor cells generated by a method of the disclosure, as described herein.

The present invention is further illustrated by the following examples, which should not be construed as further limiting. The contents of figures and all references, patents and published patent applications cited throughout this application are expressly incorporated herein by reference.

### EXAMPLES

### Example 1

### Protocol Development for the Generation of Hematopoietic Stem Cell-Derived Erythroid Progenitors

A two-stage recipe for generation of erythroid progenitors was developed that can guide human hematopoietic stem cells to progenitors expressing CD71 and CD235 after 9 days in culture. These cells can be further differentiated to enucleated erythrocytes.

This example utilizes a method of High-Dimensional Design of Experiments (HD-DoE), as previously described in Bukys et al. (2020) Iscience 23:101346. The method employs computerized design geometries to simultaneously test multiple process inputs and offers mathematical modeling of a deep effector/response space. The method allows for finding combinatorial signaling inputs that control a complex process, such as during cell differentiation. It allows testing of multiple plausible critical process parameters, as such parameters impact output responses, such as gene expression. Because gene expression provides hallmark features of the phenotype of, for example, a human cell, the method can be applied to identify, and understand, which signaling pathways control cell fate. In the current example, the HD-DOE method was applied with the intent to find conditions for induction of erythroid progenitor-expressed genes, directly from the pluripotent stem cell state.

To develop the recipe for each stage, the impact of agonists and antagonists of multiple signaling pathways (herein called effectors) on the expression of two sets of 53 pre-selected genes after a 3-day treatment has been tested and modeled. These effectors are small molecules or proteins that are commonly used during stepwise differentiation of stem cells to specific fates. The choice of the effectors was based on current literature on red blood cell (RBC) induction and differentiation of HSCs to hematopoietic progenitors, combined with available data related to cell fate control of other cell types.

### Stage 1 Recipe for Megakaryocyte/Erythrocyte Progenitor Cells

A recipe for the generation of GATA1-expressing bipotential progenitor cells for the megakaryocyte and erythrocyte lineages was developed that guided human hematopoietic stem cells (HSCs) to progenitors expressing CD34 and CD71, with less than 10% of cells positive for the downstream committed lineage markers, CD41 and CD235 (megakaryocytes and red blood cells lineage markers, respectively). These GATA1-expressing bipotential progenitor cells can be further differentiated to mature platelets and red blood cells upon further application of cell fate guidance molecules and signals. Another name of the GATA1-expressing bipotential progenitor is Megakaryocyte/Erythrocyte Progenitor (MEP) cells and the recipe for their generation is referred to herein as the Stage 1 recipe.

To test the effectors, experiments with at least 8 factors were designed that can assess the response of cells to different combinations of effectors in a range of concentrations. To analyze the models, we focused on expression of genes expressed in megakaryocyte/erythrocyte progenitors such as GATA1, TFRC, TAL1 and GATA2 among others. GATA1 null mice die at embryonic day 10 due to anemia (Pevny et al. (1991) Nature 349:257-260). GATA1 is also required for maturation of other blood lineages such as megakaryocytes, eosinophils and mast cells (Dore and Crispino (2011) Blood 118:231-239). Single cell studies of primary human CD34 cells have shown that GATA1 is low in common myeloid progenitor and highly expressed in MEP cells. Additionally, Tal1 is also required for blood formation during embryonic life (Shivdasani et al. (1995) Nature 373:432-434). GATA2 deletion in mice results in severe anemia and death at embryonic day 10 (Tsai et al. (1994) Nature 371:221-226). GATA2 is also involved in megakaryocyte development as its upregulation, inhibits erythroid differentiation and promotes megakaryopoiesis (Ikonomi et al. (2000) Exp Hematol. 28:1423-1431). The impact of each effector on gene expression level is defined by a parameter called factor contribution that is calculated for each effector during the modeling. All the experiments were conducted using a CD34⁺ population of cells isolated from cord blood.

To identify the recipe of stage 1 of differentiation, cells were grown for 3 days in media containing stem cell factor (SCF). Then, 48 different combinations of effectors generated using Design-of-Experiments compression through D-optimality were robotically prepared. The effector combinations were prepared in a basal media and were subsequently added to the cells, which were then allowed to differentiate. Three days later RNA extraction was performed and gene expression was obtained using quantitative PCR analysis. The data were normalized and modeled using partial least squares regression analysis to the effector design, resulting in the generation of gene-specific models, which after model tuning for maximal Q2 predictive power, provided an explanation of the effectors ability to control the expression of individual genes, combinatorially, and individually. Solutions within the tested space could then be explored to address desirability.

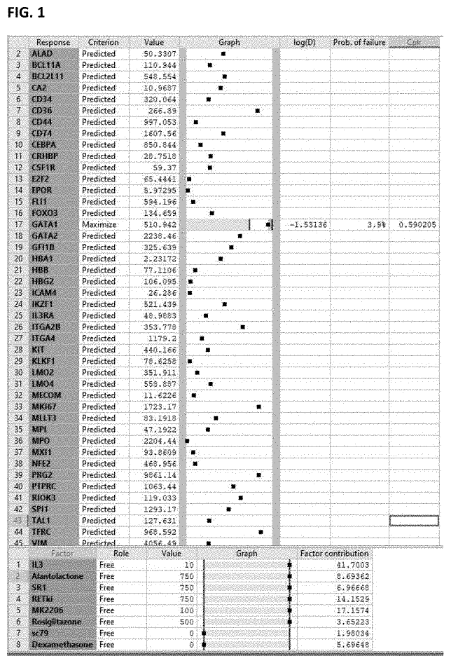

Optimizing for maximal expression of the GATA1 key regulatory gene led to a robust solution. At this solution, other genes such as MKI67, CD36, TFRC and PRG2, were also predicted to be abundantly expressed. These genes further substantiate the likelihood of this condition leading to generation of bipotential GATA1-expression progenitor cells. This model was derived from initial testing of eight factors including IL3, Alantolactone, SR1, RETki, MK2206, Rosiglitazone, sc79 and dexamethasone. As shown in FIG. 1, five of these effectors: IL3, alantolactone, SR1, RETki and MK2206 showed positive impact on expression of genes of interest with 41, 8, 6, 14 and 17 factor contributions, respectively. Within the specifications of attaining 80% maximal expression of GATA1, this complex media composition had a Cpk value (process capability index) of 0.53, with a corresponding to a 3.9% risk of failure. The invention of a complex set of inputs for GATA1-expressing MEP cells was compared to existing literature and found to be distinct. Of note, a commonly used factor in erythropoietic induction, Dexamethasone; a glucocorticoid receptor agonist, analogous to hydrocortisone, was not included, and interfered with the maximal expression of GATA1.

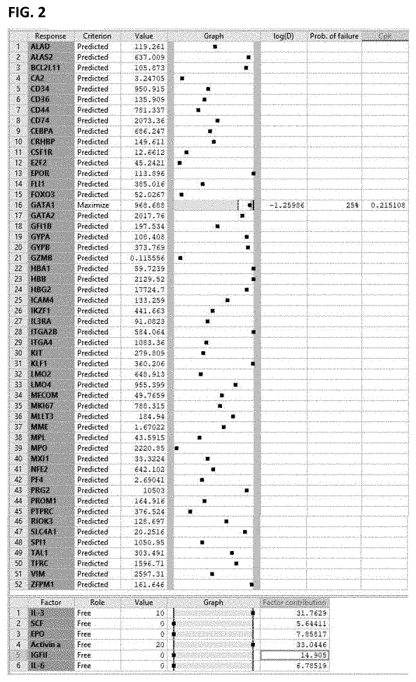

To test whether EPO or other commonly used cytokines are required for GATA1 induction, we conducted experiments with 6 factors in a range of concentrations. On this experiment CD34 cells were thawed and kept overnight in media containing SCF 10 ng/mL. On the next day, 48 different combinations of effectors were added to the cells, and SCF 5 ng/mL was kept in all the wells. Optimizing for maximal expression of the GATA1 key regulatory gene led to a robust solution. At this solution, other genes expressed in megakaryocytes and erythrocytes, such as ITGA2b, ZFPM1, KLF1 and EPOR were also predicted to be abundantly expressed. This model was derived from initial testing of IL6, IL3, SCF, EPO, Activin A and IGFII. As shown in FIG. 2, two of these effectors: IL3 and Activin A showed positive impact on expression of genes of interest with 31 and 33 factor contributions, respectively. EPO, another commonly used agent during initiation of erythropoiesis from HSC, was not required for GATA1 induction, therefore was excluded from our recipe. Activin A, which is a member of the TGFβ family, was not used in our recipe, because Alantolactone, which is also a TGFβ agonist, was supportive of GATA1 induction and is less expensive.

In view of the foregoing, a representative recipe for Stage 1 differentiation is summarized below in Table 1.

TABLE 1
Validated effectors resulting in MEP Cells from HSCs

| Effectors | Role | Concentration | |
|---|---|---|---|
| IL-3 | IL3R agonist | 10 | ng/mL |
| Alantolactone | TGFB agonist | 750 | nM |
| SR1 | AHR antagonist | 750 | nM |
| RETki | RET inhibitor | 750 | nM |
| MK2206 | AKT inhibitor | 100 | nM |

### Stage 2 Recipe for CD71+CD235+CD34− Progenitor Cells

To engineer the stage 2 media, first CD34+ cells from human cord blood cells were cultured for 5 days in stage 1 media (Table 1), which converts hematopoietic stem cells to a GATA1 bipotential progenitor (MEP progenitor). This progenitor has the potential to differentiate to erythroid and megakaryocyte lineages. To further guide the differentiation of GATA1 bipotential progenitor, an additional HD-DoE experiment was performed. Thus, additional gene regulatory models were obtained that were used for preparation of the differentiation protocol. The basis of this was a 12-factor HD-DoE experiment with focus on differentiation of cells toward the erythroid lineage for an additional 3 days after termination of stage 1 treatment.

To test the effectors, 96 different combinations of effectors generated using Design-of-Experiments compression through D-optimality were robotically prepared. The effector combinations were prepared in a basal media and were subsequently added to the cells, which were then allowed to differentiate. Three days later RNA extraction was performed, and gene expression was obtained using quantitative PCR analysis. The data were normalized and modeled using partial least squares regression analysis to the effector design, resulting in the generation of gene-specific models, which after model tuning for maximal Q2 predictive power, provided explanation of the effectors ability to control the expression of individual genes, combinatorically, and individually. Solutions within the tested space could then be explored to address desirability.

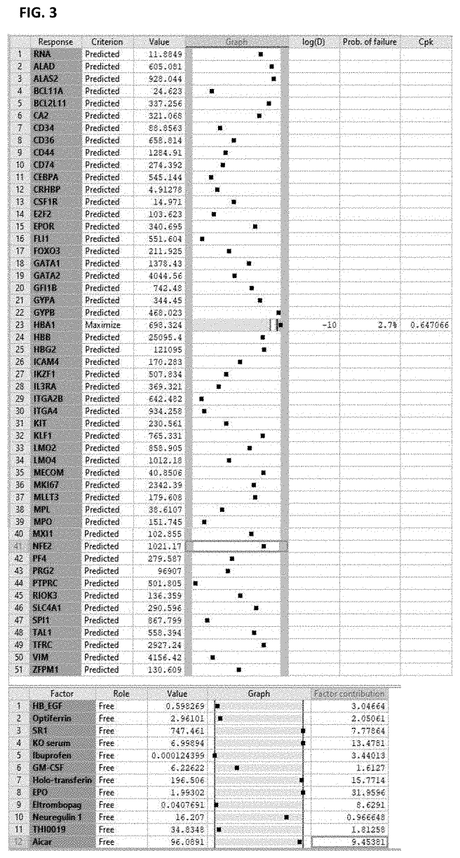

Optimizing for maximal expression of the HBA1 led to a robust solution. At this solution, other genes were also predicted to be abundantly expressed, such as MKI67 TFRC, HBB, HBG2, ALAD, ALAS2, CA2, GYPA, GYPB, GATA1, KLF1 and TFRC, all genes highly expressed on erythroid cells, suggesting cell commitment to this lineage. On the same model, CSF1R, TPOR and MPO were downregulated (gene associated to other hematopoietic lineages). Additionally, genes related to the stem cell state, CD34, MECOM and CRHBP, were downregulated, indicating that cells are differentiating. This model was derived from initial testing of twelve factors including: HB-EGF, Optiferrin, SR1, knockout serum, ibuprofen, GM-CSF, holo-transferrin, EPO, eltrombopag, Neuregulin 1, THI0019 and AICAR. As shown in FIG. 3, five of these effectors: SR1, knockout serum, holotransferin, EPO, and AICAR showed positive impact on expression of genes of interest with 7, 13,15, 31 and 9 factor contributions, respectively. Within the specifications of attaining 80% maximal expression of HBA1, this complex media composition had a Cpk value (process capability index) of 0.64, with a corresponding to a 2.7% risk of failure.

In view of the foregoing, a representative recipe for Stage 2 differentiation is summarized below in Table 2.

TABLE 2
Validated Effectors in Stage 2 Recipe for CD71+CD235+CD34−
Progenitor Cells

| Effectors | Role | Concentration | |
|---|---|---|---|
| SR1 | AHR antagonist | 750 | nM |
| Holotransferrin | Iron source | 200 | ug/mL |
| EPO | EPOR agonist | 2 | U/mL |
| AICAR | AMPK agonist | 100 | uM |

| | | |
|---|---|---|
| Albumax | Lipids source | 0.5% |

### Example 2

### Factor Criticality Analysis of Hematopoietic Stem Cell-Derived Erythroid Progenitor-Inducing Culture Conditions

To assess the impact of the elimination of each validated factor identified for the stage 1 and 2 recipes as described in Example 1, dynamic profile analysis was used and compared the expression level of genes of interest in absence of each finalized factor while others are present. Since expression levels of genes of interest reveal whether the desired outcome is reachable, this factor criticality analysis revealed the extent of importance of each input effector.

### Stage 1 Recipe

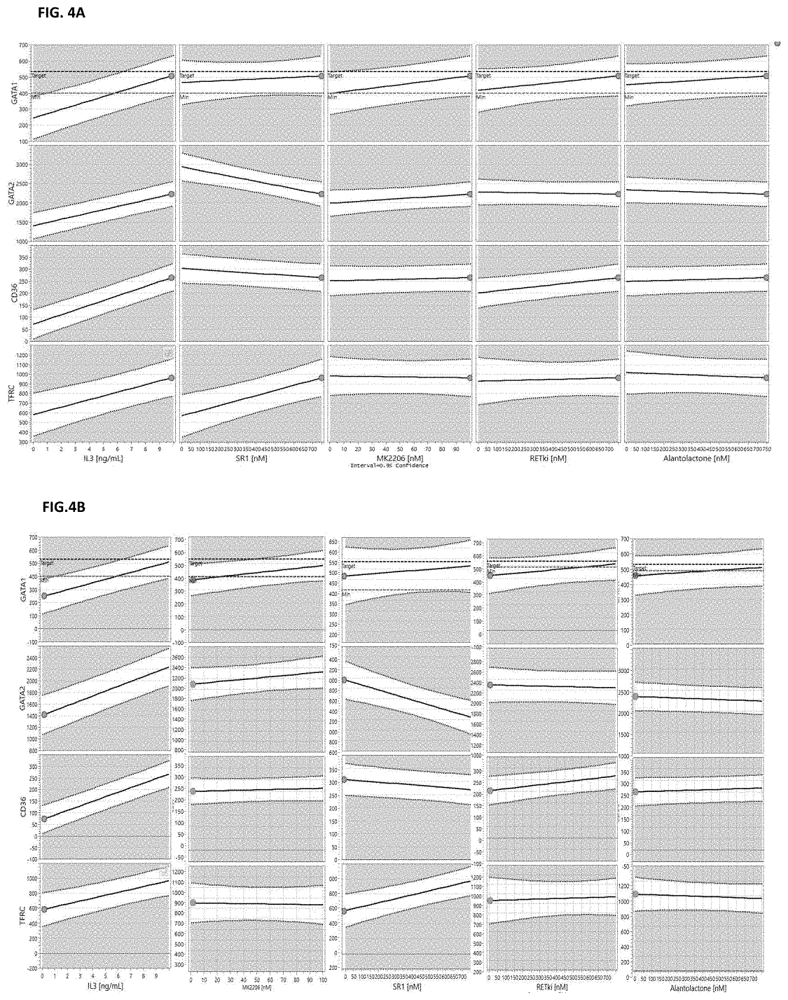

In the stage 1 recipe, each of the five finalized factors was removed while the other four factors remained present and the expression levels of GATA2, GATA1, CD36 and TFRC were assessed compared to the presence of all five factors. The results are summarized in FIGS. 4A-4B. When IL-3 was removed, the values of GATA1 expression decreased from 510 to 240, the values of CD36 expression changed from 260 to 70, the values of GATA2 expression decreased from 2200 to 1400 and the values of TFRC expression dropped from 960 to 580. All changes represent a significant loss of expression of a desired gene for the MEP progenitor.

The absence of SR1 resulted in reduced expression of GATA1, a decrease from 510 to 410; SR1 also was noted to be a critical factor for TFRC expression since its removal decreased levels from 970 to 570. MK2206 was involved in securing maximal GATA1 expression, wherein removal of this AKT inhibitor decreased levels of GATA1 from 510 to 400. The absence of RETki resulted in reduced expression of GATA1, a decrease from 510 to 410. When Alantolactone was removed, values of GATA1 decreased from 510 to 460, having less of an impact on GATA1 expression as the others.

### Stage 2 Recipe

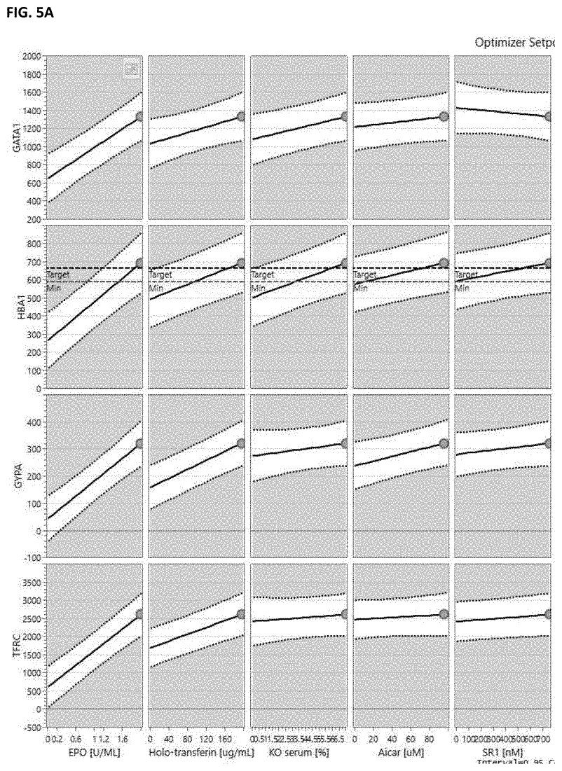

In the stage 2 recipe, each of the five finalized factors was removed while the other four factors remained present and the expression levels of GATA1, HBA1, GYPA (CD235) and TFRC were assessed compared to the presence of all five factors. The results are summarized in FIGS. 5A-5B. When EPO was removed, the values of GATA1 decreased from 1386 to 736, the values of HBA1 changed from 700 to 285, the values of GYPA decreased from 345 to 61 and the values of TFRC dropped from 2943 to 898. All changes represent a significant loss of expression of a desired gene.

When holotransferrin was removed, the values of GATA1 decreased from 1370 to 967, the values of HBA1 decreased from 688 to 500, the values of GYPA decreased from 330 to 170 and the values of TFRC decreased from 2883 to 1703. Knockout serum was found to be critical for HBA1 and GATA1 induction, since removal of this factor decreased HBA1 expression from 688 to 520 and GATA1 expression changed from 1357 to 1221.

AICAR, a AMPK agonist, was found to be a critical factor for upregulation of HBA1 and GYPA. When AICAR was removed, the levels of HBA1 dropped from 689 to 569, and the levels of GYPA decreased from 337 to 256. SR1 addition has an impact on HBA1 and glycophorins A and B expression. HBA1 expression decreased from 700 to 597 when SR1 was removed.

### Example 3

### Flow Cytometry Validation of Cell Culture Media for Induction of Hematopoietic Stem Cell-Derived Erythroid Progenitor Cells

To validate the developed recipes described in example 1, cells were treated with stage 1 and stage 2 media, versus a literature media, and flow cytometry was used to assess biomarker expression at the end of each stage.

### Stage 1 Recipe

To validate the stage 1 recipe described in example 1, CD34+ cord blood derived cells were grown for 4 and 5 days in stage 1 GATA1 bipotential progenitor inducing media, and flow cytometry analysis was used to assess expression of hematopoietic progenitor markers. Comparatively, cells were grown in differentiation media described in the literature, commonly used to promote RBC differentiation (IL3 10 ng/mL, hydrocortisone 1 uM, SCF 100 ng/mL and EPO 6U/mL) in order to compare with the stage 1 GATA1-expressing bipotential progenitor media.

Based on the literature, different combinations of markers can be used to identify megakaryocyte/erythrocyte progenitors (MEP cells) such as Lin⁻CD34⁺CD38^{mid/low}/CD45RA⁻ (Sanada et al. (2016) Blood 128:923-933; Debili et al. (1996) Blood 88:1284-1296) or alternatively Lin⁻CD34⁺CD38⁺CD123⁻CD45RA⁻ (Cimato et al. (2016) Cytometry B. Clin. Cytom. 90:415-423). Expression of all the markers mentioned above were assessed and complemented with terminal markers for the megakaryocyte and erythrocyte lineages as well. Of these markers, CD34 is expressed in the original HSC population and its expression decreases during differentiation into either lineage. Additional markers evaluated were: CD41; a megakaryocyte marker also known as Integrin A2B (ITGA2B); CD235 (glycophorin) which is exclusively expressed by red blood cells; and CD71 (TRFC, Transferrin receptor C) expression of which increases substantially during erythrocyte differentiation but is also expressed during the GATA1 expressing progenitor stage. Complete differentiated red blood cells either lack or express low levels of CD71.

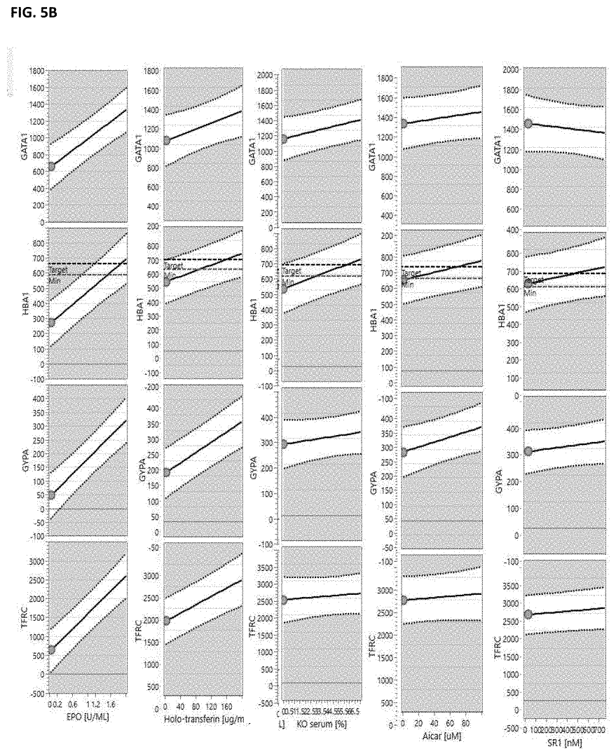

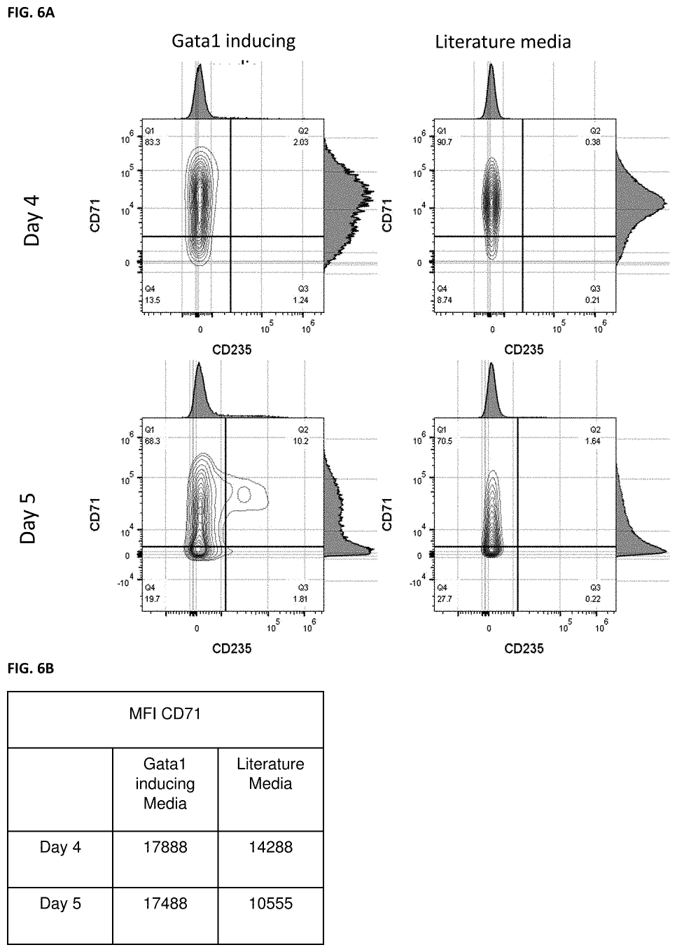

After growing the cells for 4 and 5 days, flow cytometry analysis was performed and evaluated the immunophenotype of CD34 cells grown in the GATA1+ bi-potential cell media. Flow cytometry analysis confirmed the efficiency of the recipe to promote conversion of HSC to progenitors. As shown in FIGS. 6A-6B, the relative fraction of cells that are CD71+ using either media is comparable in day 4 and 5. CD71 mean fluorescence intensity (MFI) reflects the expression level of the target and the stage 1 media led to significant increase in protein expression. Both on day 4 and day 5, CD71 levels were higher in stage 1 media compared to literature media. As shown in FIG. 6A, glycophorin expression, as denoted by CD235 and a terminal commitment marker of the erythrocyte, was low on day 4 on both medias, however on day 5, ˜10% of cells were CD235+ using stage 1 media compared to 1.64% of cells that were CD235+ using literature media, which suggested that the stage 1 media can accelerate the differentiation to the committed erythroid lineage.

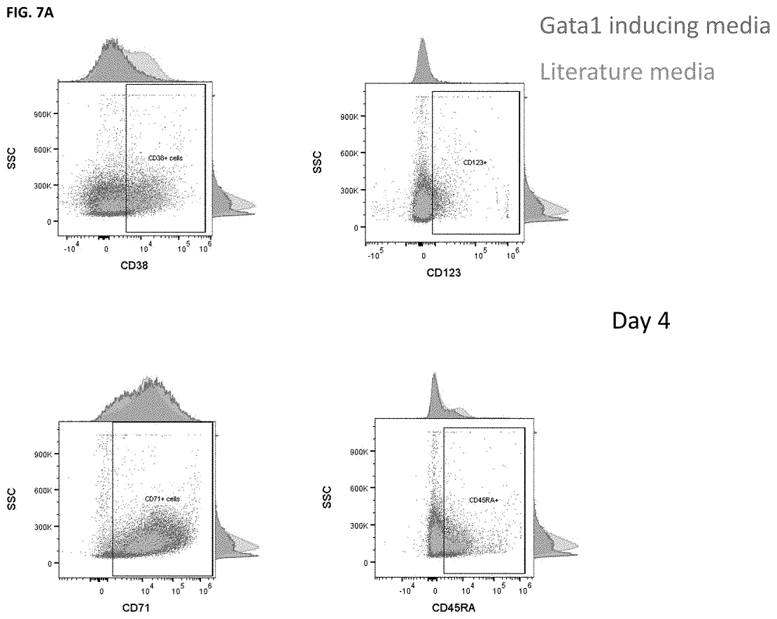

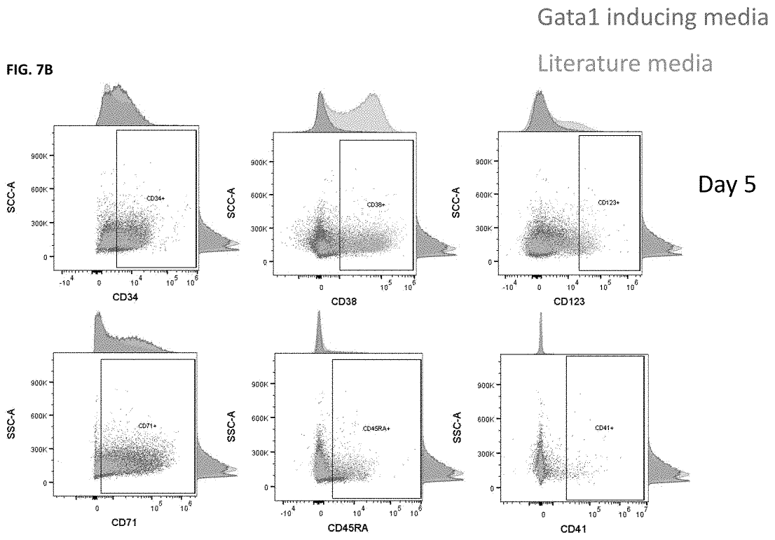

FIGS. 7A-7B show CD34, CD38, CD123, CD71, CD45RA and CD41 staining on cells grown using stage 1 media (red) and literature media (blue) for 4 and 5 days. CD41/ITGA2B levels, a commitment marker for the platelet lineage, were low or absent on both conditions at day 5. On day 5, CD34 levels were similar in both conditions, suggesting retention of progenitor attributes. CD38 expression was comparatively higher using literature media cells on day 4 and 5. Previous studies (Sanada et al., 2016; Debili et al., 1996) have shown that erythroid progenitors are CD38+, while bipotent progenitor (Megakaryocyte/Erythrocyte Progenitors) are low or negative for CD38. FIGS. 7A-7B also show that the majority of cells are CD45RA and CD123 negative using either media (CD45RA: ˜15% literature media versus ˜11% stage 1 media on day 5; CD45RA: 39% literature media versus 28% stage 1 media on day 4; CD123: 7.6% literature media versus 1.1% on day 5 stage 1 media; CD123: ˜6.6% literature media versus ˜10% on stage 1 media at day 4). These markers reveal the absence of induction of interfering myeloid lineages using these induction conditions, granulocyte/monocyte progenitor cells are CD45RA⁺ and the common myeloid progenitor and granulocyte monocyte progenitor are both CD123+.

### Stage 2 Recipe

To validate the stage 2 recipe described in example 1, CD34+ cord blood derived cells were grown for 4 days in stage 1 media, and then grown for 3, 5 and 7 days on stage 2 media and flow cytometry analysis was used to assess expression of erythroid markers. Because knockout serum has a proprietary formula and is expansive, we also tested the same recipe, removing knockout serum and adding 0.5% albumax instead, which is the main component of knockout serum. Albumax is bovine serum albumin loaded with lipids. Additionally, cells were grown using a literature media for differentiation of RBCs (Stage 1: IL3 10 ng/mL, hydrocortisone 1 uM, SCF 100 ng/mL and EPO 6 U/m; Stage 2: SCF 50 ng/mL, EPO 3 U/mL and 200 ug/mL holotransferrin) in order to compare with the stage 2 media. The expression of CD34 (which is expressed in HSCs, with expression decreasing during differentiation) was also assessed, while CD235 is expressed by red blood cells. CD71 expression increases substantially during RBC differentiation, however it decreases during later stages of differentiation.

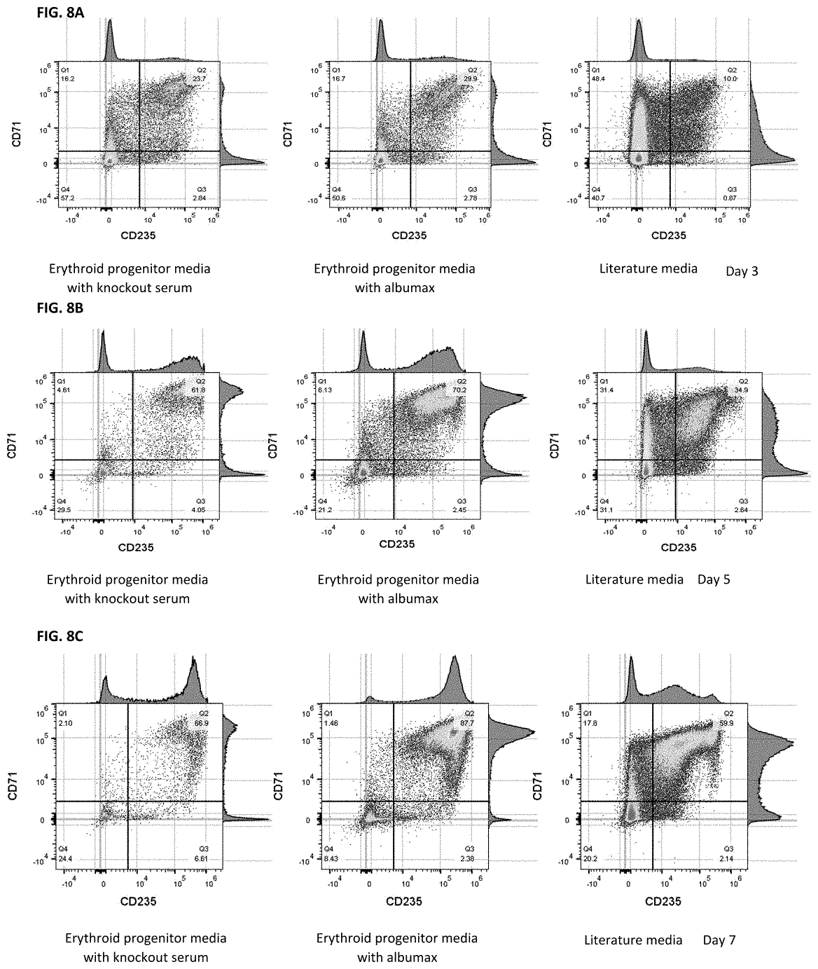

As shown in FIGS. 8A-8C, flow cytometry analysis confirmed the efficiency of the stage 2 media to promote conversion of the MEP cell to erythroid progenitors. More importantly, the stage 2 media containing albumax promoted faster differentiation (higher CD235 expression) when compared to the literature recipe. The results in FIG. 8A show that on day 3 the percentage of cells expressing CD71 and CD235 using literature media is lower compared to the stage 2 media (10% versus 23% and 29%). As shown in FIG. 8B, on day 5 the stage 2 media promoted faster differentiation when compared to the literature media. At this time point 70% of the cells were CD71 and CD235 double positive on the stage 2 media versus 34% on the literature media. Finally, as shown in FIG. 8C, on day 7, the stage 2 media again performed better than the literature media, resulting in 87% of cells being CD71 and CD235 double positive versus 59% on the literature media. Altogether, this data shows that the stage 2 media is faster, promoting a more efficient conversion to the erythroid lineage, and less expensive than the literature protocol.

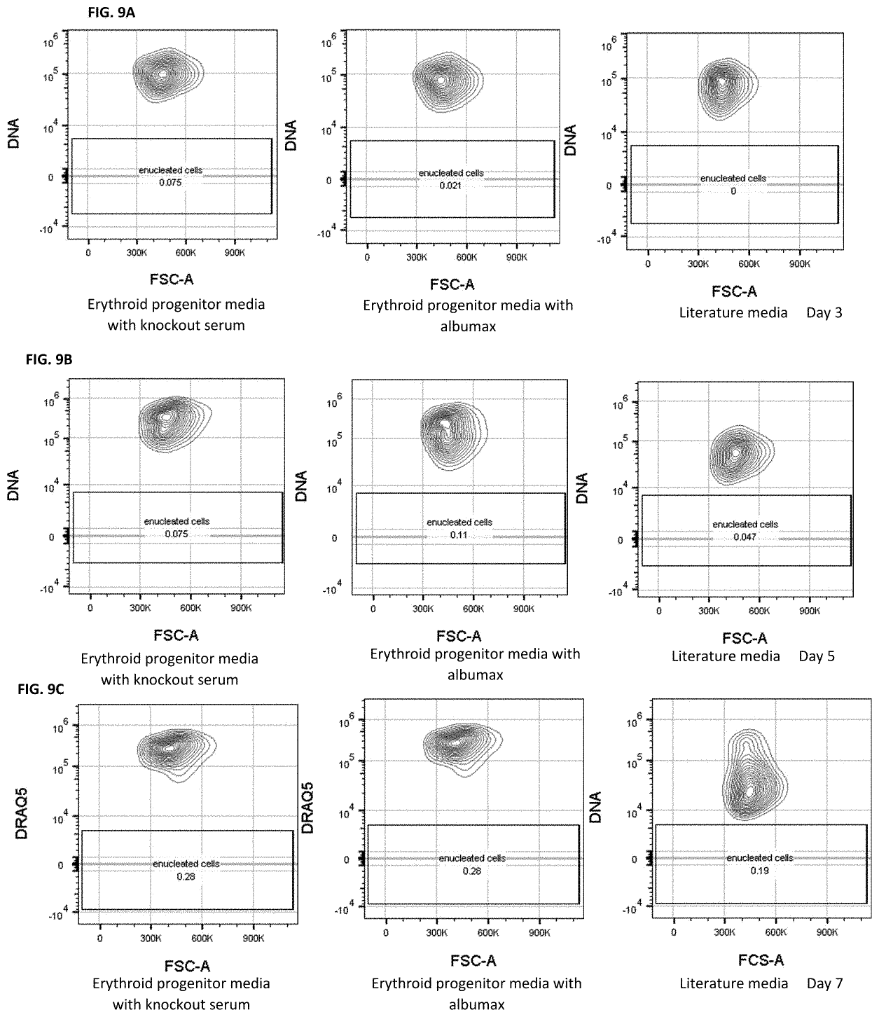

Enucleation is one of the final steps of human RBC differentiation. To measure cell enucleation during stage 2, the cell permeable DNA dye DRAQ5 was used, followed by performance of flow cytometry analysis. The results are shown in FIGS. 9A-9C. As expected, since it is too early in the process of differentiation, no enucleation was observed in any of the conditions tested.

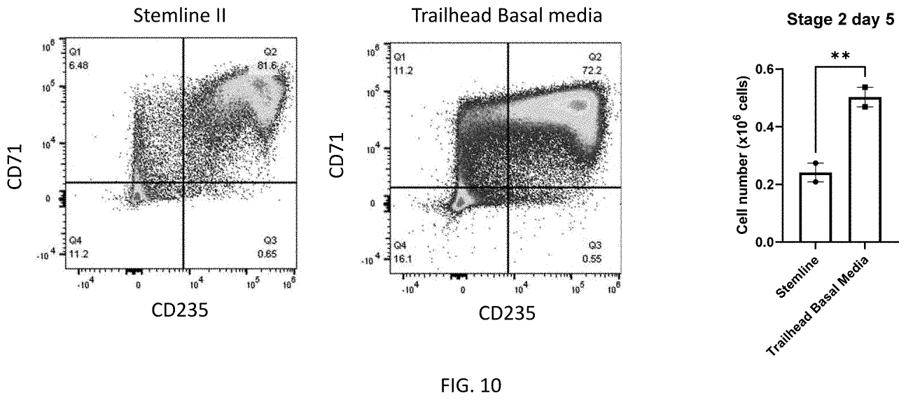

To develop a cost-effective basal media for RBC differentiation, different basal media compositions were tested and compared to Stemline II performance. The performance of the basal media was tested during stage 2 differentiation (our stage 2 recipe). One basal media composition in particular showed similar performance to StemLine II regarding differentiation. The components of this basal media are shown below in Table 3. As demonstrated in FIG. 10, which shows flow cytometry analysis of CD71 and CD235, the differentiation profiles in both medias were similar; however two times more proliferation was observed using the exemplary basal media of Table 3 as compared to Stemline II media.

TABLE 3
Composition of Exemplary Basal Media

| Components | Final Conc. | |
|---|---|---|
| Albumax | 1% | |
| Ethanolamine | 10 | uM |
| Monothioglycerol | 150 | uM |
| Cholesterol sulfate | 409 | nM |
| Trolox | 10 | uM |
| Linoleic acid | 4 | ng/mL |
| Oleic acid | 50 | uM |
| Insulin | 10 | ug/mL |

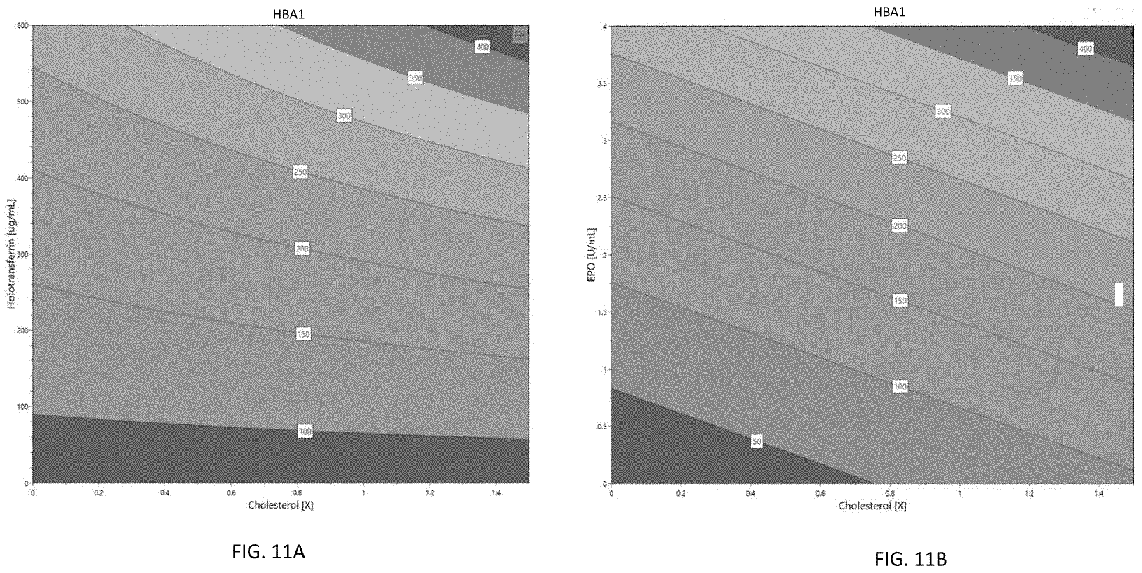

In order to refine the stage 2 recipe, more modelling experiments were conducted using the same factors as the stage 2 recipe but testing different concentrations of critical factors such as EPO and holo-transferrin. Additionally, because lipid inputs are critical for RBC differentiation, a commercial cholesterol supplement (Sigma) was tested. Results are shown in FIGS. 11A-11B. Optimization for maximal expression of HBA showed that higher concentrations of holo-transferrin (600 ug/mL) and EPO (4 U/mL) led to higher HBA expression. Cholesterol supplement also had a positive impact on HBA expression. Quantification of cell number showed that addition of cholesterol supplement, and increasing EPO (4 U/mL) and holo-transferrin (600 ug/mL) on the stage 2 recipe promoted higher proliferation when compared to control.

### Example 4

### Differentiation Potential of Hematopoietic Stem Cell-Derived Megakaryocyte/Erythroid Progenitor Cells

In order to examine the differentiation potential of the MEP cells, CD34+ HSCs were cultured for 4 days in stage 1 media (as described in Example 1). Then, using HD-DoE as described in Example 1, 96 different combinations of effectors (12 in total) were generated in basal media which were added to the cells, and cells were allowed to further differentiate. Three days later, RNA extraction was performed, and gene expression was modeled as previously described. 12 factors were tested: HB-EGF, Optiferrin, SR1, knockout serum, ibuprofen, GM-CSF, holo-transferrin, EPO, PD102807, Neuregulin 1, THI0019 and AICAR in this lineage challenge experiment. Gene specific models were obtained analogously to earlier description, and similarly interrogated for optimal conditions related to induction of downstream lineages.

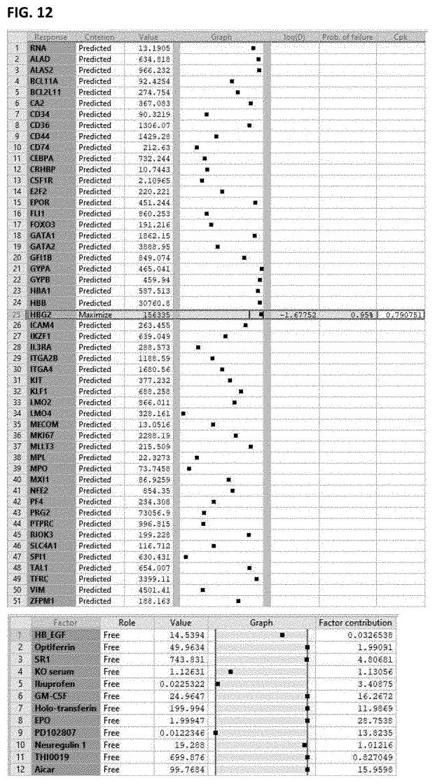

To analyze the models, we focused on expression of genes expressed in committed erythroid progenitors and fully differentiated erythrocytes, such as HBG2, HBA, HBB, GYPA, GYPB, EPOR, ALAD, TFRC among others. Of these genes, HBG2, HBA, HBB encode hemoglobins; GYPA, GYPB, glycophorins, EPOR (EPO receptor), ALAD (encoding the rate limiting enzyme for heme synthesis in the erythrocyte). One model, the results of which are summarized in FIG. 12, revealed upregulation of all the genes mentioned above, plus CA2, ALAS2, GATA1, ICAM4, TAL1 and MLLT3, multiple of which were previously demonstrated to be associated with the erythrocyte lineage. This experiment demonstrated the ability of the MEP cells obtained using the stage 1 media to commit to the erythroid lineage and activate terminal genes. On this model we showed elevated expression of hemoglobins, with HBG2 expression around 156335, which after normalization represent extremely abundant hemoglobin mRNA production.

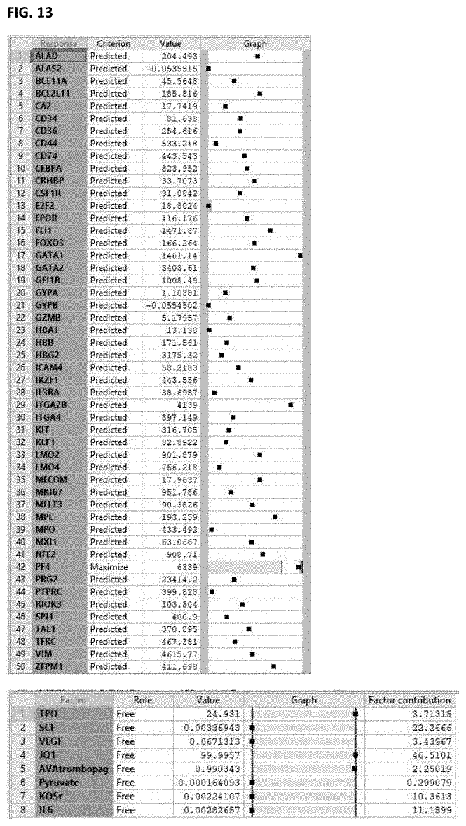

Next a similar experiment to the one described above was performed challenging the cells towards the megakaryocyte/platelet lineage; however, 8 factors were used instead of 12 resulting in 48 different combinations of effectors. Factors included pathway regulatory inputs that are commonly used to induce platelet differentiation, enhanced with others that we hypothesize would control the platelet lineage: TPO, SCF, VEGF, JQ1, Avatrombopag, pyruvate, knockout serum replacement media additive, and IL6. To analyze the models, we focused on expression of genes expressed in megakaryocyte progenitors such as PF4, ITGA2b, FLI1, ZFPM1. One model, the results of which are summarized in FIG. 13, specifically, revealed significant upregulation of all the genes mentioned, suggesting commitment to the platelet lineage. Based on this model, high expression of PF4, around 6339, was observed, and other known megakaryocyte expressed genes were upregulated as well, demonstrating full commitment with the megakaryocyte lineage. Altogether this data shows that HSCs grown on stage 1 media for 4 days generate MEP cells that have the potential to differentiate into two lineages: the erythroid lineage and the megakaryocyte lineage, dependent on subsequent lineage-inducing inputs.

Equivalents

Those skilled in the art will recognize, or be able to ascertain using no more than routine experimentation, many equivalents of the specific embodiments of the invention described herein. Such equivalents are intended to be encompassed by the following claims

1. A method of generating human GATA1+ megakaryocyte/erythroid progenitor (MEP) cells comprising: culturing human CD34+ hematopoietic stem cells (HSCs) in a culture media comprising an IL3R pathway agonist, a TGFβ pathway agonist, an AHR pathway antagonist, a RET pathway antagonist and an AKT pathway antagonist on days 0-4 to obtain human GATA1+ MEP cells.

2. The method of claim 1, wherein the human GATA1+ MEP cells are further cultured on days 4-9 in a culture media comprising an AHR antagonist, an iron source, an EPOR agonist, an AMPK agonist and a lipid source to obtain human CD71+CD235+CD34− erythroid progenitor cells.

3. The method of claim 1, wherein the HSCs are from umbilical cord blood.

4. The method of claim 1, wherein the HSCs are from bone marrow or peripheral blood.

5. The method of claim 1, wherein the IL3R pathway agonist is IL-3.

6. The method of claim 5, wherein IL-3 is present in the culture media at a concentration within a range of 5-15 ng/ml.

7. The method of claim 5, wherein IL-3 is present in the culture media at a concentration of 10 ng/ml.

8. The method of claim 1, wherein the TGFβ pathway agonist is selected from the group consisting of alantolactone, Activin A, TGFB1 and Nodal and combinations thereof.

9. The method of claim 8, wherein the TGFβ pathway agonist is present in the culture media at a concentration within a range of 500-1000 nM.

10. The method of claim 8, wherein the TGFβ pathway agonist is alantolactone, which is present in the culture media at a concentration of 750 nM.

11. The method of claim 1, wherein the AHR pathway antagonist is selected from the group consisting of SR1, GNF351, AHR antagonist 5 hemimaleate, AHR antagonist 1, PDM2, BAY 2416964, CH-223191, AHR antagonist 2, AHR antagonist 4, and combinations thereof.

12. The method of claim 11, wherein the AHR pathway antagonist is present in the culture media at a concentration within a range 500-1000 nM.

13. The method of claim 11, wherein the AHR pathway antagonist is SR1, which is present in the culture media at a concentration of 750 nM.

14. The method of claim 1, wherein the RET pathway antagonist is selected from the group consisting of RETki, Lenvatinib, Regorafenib, Pralsetinib, Selpertinib, Lenvatinib mesylate, RET-IN-4, RPI-1, JNJ38158471, Amuvatinib, TG101209, Regorafenib Hydrochloride, Ilorasertib hydrochloride, AST487, PF477736, BBT594, AD80, GSK3179106, SPP86, RET-IN-3, WF-47-JS03, RET V804M-IN-1, Trans-pralsetinib, PZ1, Regorafenib D3, RET-IN-1, ML786 dihydrochloride, WHI-P180 hydrochloride, and combinations thereof.

15. The method of claim 14, wherein the RET pathway antagonist is present in the culture media at a concentration within a range of 500-1000 nM.

16. The method of claim 14, wherein the RET pathway agonist is RETki, which is present in the culture media at a concentration of 750 nM.

17. The method of claim 1, wherein the AKT pathway antagonist is selected from the group consisting of MK2206, GSK690693, Perifosine (KRX-0401), Ipatasertib (GDC-0068), Capivasertib (AZD5363), PF-04691502, AT 7867, Triciribine (NSC154020), ARQ751, Miransertib (ab235550), Borussertib, Cerisertib, Akti1/2, CCT128930, A 674563, PHT 427, Miltefosine, AT 13148, ML 9, BAY 1125976, Oridonin, TIC10, Pectolinarin, Acti IV, 10-DEBC, API-1, SC 66, FPA 124, API-2, Urolithin A, and combinations thereof.

18. The method of claim 17, wherein the AKT pathway antagonist is present in the culture media at a concentration within a range of 50-150 nM.

19. The method of claim 17, wherein the AKT pathway antagonist is MK2206, which is present in the culture media at a concentration of 100 nM.

20. The method of claim 2, wherein the iron source is selected from the group consisting of holotransferrin, FeIII_EDTA, Optferrin, FeSO4, Ferrous nitrate, lactoferrin, ferritin, and combinations thereof.

21. The method of claim 20, wherein the iron source is present in the culture media at a concentration within a range of 150-250 ug/ml.

22. The method of claim 20, wherein the iron source is holotransferrin, which is present in the culture media at a concentration of 200 ug/ml.

23. The method of claim 2, wherein the EPOR pathway agonist is selected from the group consisting of, and combinations thereof.

24. The method of claim 23, wherein the EPOR pathway agonist is EPO.

25. The method of claim 23, wherein the EPOR pathway agonist is EPO, which is present in the culture media at a concentration of 2 U/ml.

26. The method of claim 2, wherein the AMPK pathway agonist is selected from the group consisting of AICAR, Metformin, BC1618, Malvidin-3-O-arabinoside chloride, A-769662, MK8722, Bempedoic acid, AICAR phosphate, Phenformin hydrochloride, EX229, gingerol, Kazinol B, PF06409577, Flufenamic acid, GSK621, Urolithin B, MK3903, chitosan oligosaccharide, palmitelaidic acid, O-304, Amarogentin, 7-Methoxyisoflavone, EB-3D, Buformin hydrochloride, Platycodin D, ZLN024 hydrochloride, Danthron, Ampkinone, ginkolide C, Gomisin J, Demethylenebernerine, ASP4132, IM156, Vacarin, MOTS-c(human) acetate, Kahweol, AMPK activator 4, Marein, Euphorbiasteroid, Cimiracemoside C, Metformin D6 hydrochloride, MT6378, RSVA405, Nepodin, 3α-Hydroxymogrol, AMPK activator 1, YLF-466D, Buformin, IQZ23, Galegine hydrochloride, Karanjin, COH-SR4, HL271, ZLN024, EB-3D, and combinations thereof.

27. The method of claim 26, wherein the AMPK pathway agonist is present in the culture media at a concentration within a range of 50-150 uM.

28. The method of claim 26, wherein the AMPK pathway agonist is AICAR, which is present in the culture media at a concentration of 100 uM.

29. The method of claim 2, wherein the lipid source is selected from the group consisting of Albumax, free fatty acids, lysophosphatidylcholine triacylglycerides, phosphatidylcholine, phosphatidic acid, cholesterol, sphingomyelin, knockout serum replacement, Lipid Mixture 1™, Chemically Defined Lipid Concentrate™, bovine serum albumin, human serum albumin, and combinations thereof.

30. The method of claim 29, wherein the lipid source is Albumax.

31. The method of claim 29, wherein the lipid source is Albumax, which is present in the culture media at a concentration of 0.5%.

32. A method of generating human CD71+CD235+CD34− erythroid progenitor cells, the method comprising:
(a) culturing human CD34+ hematopoietic stem cells (HSCs) in a culture media comprising an IL3R pathway agonist, a TGFβ pathway agonist, an AHR pathway antagonist, a RET pathway antagonist and an AKT pathway antagonist on days 0-4 to obtain human GATA1+ MEP cells; and
(b) further culturing the human GATA1+ MEP cells in a culture media comprising an AHR antagonist, an iron source, an EPOR agonist, an AMPK agonist and a lipid source on days 4-9 to obtain human CD71+CD235+CD34− erythroid progenitor cells.

33. The method of claim 32, wherein in step (a) the IL3R pathway agonist is IL-3, the TGFβ pathway agonist is alantolactone, the AHR pathway antagonist is SR1, the RET pathway antagonist is RETki, and the AKT pathway antagonist is MK2206.

34. The method of claim 33, wherein in step (a) IL-3 is present in the culture media at a concentration of 10 ng/ml, alantolactone is present in the culture media at a concentration of 750 nM, SR1 is present in the culture media at a concentration of 750 nM, RETki is present in the culture media at a concentration of 750 nM, and MK2206 is present in the culture media at a concentration of 100 nM.

35. The method of claim 32, wherein in step (b) the AHR pathway antagonist is SR1, the iron source is holotransferrin, the EPOR pathway agonist is EPO, the AMPK pathway agonist is AICAR, and the lipid source is Albumax.

36. The method of claim 35, wherein in step (b) SR1 is present in the culture media at a concentration of 750 nM, holotransferrin is present in the culture media at a concentration of 200 ug/ml, EPO is present in the culture media at a concentration of 2 U/ml, AICAR is present in the culture media at a concentration of 100 uM, and Albumax is present in the culture at a concentration of 0.5%.

37. A culture media for obtaining human GATA1+ megakaryocyte/erythroid progenitor (MEP) cells comprising an IL3R pathway agonist, a TGFβ pathway agonist, an AHR pathway antagonist, a RET pathway antagonist and an AKT pathway antagonist.

38. A culture media for obtaining human CD71+CD235+CD34− erythroid progenitor cells comprising an AHR antagonist, an iron source, an EPOR agonist, an AMPK agonist and a lipid source.

39. An isolated cell culture of human GATA1+ megakaryocyte/erythroid progenitor (MEP) cells, the culture comprising human GATA1+ MEP cells cultured in a culture media comprising an IL3R pathway agonist, a TGFβ pathway agonist, an AHR pathway antagonist, a RET pathway antagonist and an AKT pathway antagonist.

40. An isolated cell culture of human CD71+CD235+CD34− erythroid progenitor cells, the culture comprising human CD71+CD235+CD34− erythroid progenitor cells cultured in a culture media comprising an AHR antagonist, an iron source, an EPOR agonist, an AMPK agonist and a lipid source.

41. Human GATA1+ megakaryocyte/erythroid progenitor (MEP) cells generated by the method of claim 1.

42. Human CD71+CD235+CD34− human erythroid progenitor cells generated by the method of claim 2.
